(19) United States
(12) Patent Application Publication
(10) Pub. No.: US 2003/0069545 A1
(43) Pub. Date: Apr. 10, 2003

(54) Graft delivery syringe

(76) Inventor: Douglas M. Arm, Santa Margarita, CA (US)

Correspondence Address: Robert D. Katz, Cooper & Dunham LLP, 1185 Avenue of the Americas, New York, NY, 10036, US

(21) Appl. No.: 10/233,254

(22) Filed: Aug. 28, 2002

Related U.S. Application Data
(63) Continuation of application No. 09/454,657, filed on Dec. 6, 1999, now abandoned.

(51) Int. Cl. A61B 17/58

(52) U.S. Cl. 604/218; 606/93

(57) ABSTRACT

The invention provides for dispensing a bone substitute material, comprising: an elongated body having an outer wall, a front opening, and a rear opening, with the outer wall defining a longitudinally extending interior space therein. The invention also includes a cover for the front opening having a sealable opening therein to allow substances to pass through the opening. A plunger sized to slide within the longitudinally extending cavity divides the cavity into first and second portions. The plunger preferably has a small opening therein to allow passage of fluids (including gases), from the longitudinally extending interior space without permitting solids to pass therethrough. A detachable handle connects to the plunger for sliding the plunger toward or away from the front opening.

### FIELD OF THE INVENTION

The invention relates to devices for implantation of bone replacement materials, and more particularly to a syringe which can mix and dispense solid bone substitutes and fluid additives, such as gels, suspensions or solutions of blood or its components, or natural or recombinant bone growth factors.

### BACKGROUND OF THE INVENTION

Several materials have been developed to fill a void in a bone or to cause a fusion between adjacent bones or segments of bone. Such a void may exist because a diseased portion of the bone has been surgically removed or has been damaged in an accident. The surgeon may desire to fill the space with a temporary or permanent bone substitute material, as well as with one or more agents which help to promote healing.

For example, U.S. Pat. No. 4,976,736 (Shors and White), entitled “Coated BioMaterial and Methods for Making Same” discloses a bone repair material which includes a phase which rapidly breaks down to allow tissue ingrowth, as well as a phase which resorbs more slowly. Such a material, sold commercially as Interpore 500R, often gets used as an implant material to fill bone voids or other spaces during orthopedic surgery. In granular form, it may be implanted either with or without a fluid which includes, for example, a gel, suspension, or solution of blood or its components, or natural or recombinant growth factors, for example. Mixing and handling the solid, dry granules and the fluid or gel can be difficult, particularly during an actual procedure.

Untied U.S. Pat. No. 4,769,011 (Swaniger), discloses several plunger-type syringes for the surgical implantation of granular ceramic substances including hydroxyapatite during alveolar ridge augmentation and repairs. The barrels of the syringes have various lengths and radii of curvature and various degrees of tip beveling, such that the surgeon can select the most appropriate barrel configuration for the area undergoing repair.

It would be desirable therefore to have a device which could help the surgeon dispense the granules or other particles into the desired cavity with the necessary control over the volume dispensed. Further, it would be desirable for the dispenser to have the ability to mix a porous or non-porous bone repair material (whether in block, granule, or particle form) with a liquid or gel which may contain the bone repair promoting agent.

### SUMMARY OF THE INVENTION

The objects of the invention can be achieved and the disadvantages of the prior art may be overcome through the present invention. The invention provides a syringe assembly for dispensing a bone substitute material, comprising: an elongated body having an outer wall, a front opening, and a rear opening, with the outer wall of the elongated body defining a longitudinally extending interior space therein. The invention also includes a cover for the front opening having a sealable opening therein to allow substances to pass through the opening. A plunger sized to slide within the longitudinally extending cavity divides the cavity into first and second portions. The plunger preferably has a small opening therein to allow passage of fluids (including gases), from the longitudinally extending interior space without permitting solids to pass therethrough. A detachable handle connects to the plunger for sliding the plunger toward or away from the front opening.

The invention also provides a kit for use in mixing a fluid with a porous or non-porous solid bone substitute material. The kit comprises a syringe assembly as discussed in the foregoing paragraph, and may also include a container having a solid or porous particulate or granular bone repair material, such as an optional vial or container of a bone promoting agent. The solid material may be hydroxyapatite granules, for example. The bone repair promoting agent may be in solid or liquid form, and may include (for example) blood or a component thereof, or natural or recombinant bone growth factors, or other agents useful for stimulating bone repair or growth.

### BRIEF DESCRIPTION OF THE DRAWINGS

Further features, objects, and advantages of the invention will become apparent upon review of the following detailed description of the preferred embodiments taken in conjunction with the accompanying drawings in which:

FIG. 1 is a perspective view of the syringe assembly of the present invention showing the piston handle disconnected from the piston;

FIG. 2 is a partial cross-sectional view of a syringe barrel the syringe assembly of FIG. 1 shown with the rear cap removed from the piston;

FIG. 3 is a front elevational view partially broken away of the syringe barrel shown in FIG. 2;

FIG. 4 is a top view of the piston handle of the syringe assembly of the present invention;

FIG. 5 is a side elevational view, partially in section, of the piston handle taken along line 5-5 in FIG. 4;

FIG. 6 is a cross-sectional view of the handle taken along line 6-6 in FIG. 4;

FIG. 7 is a top view of the front cap for use in the syringe assembly of the present invention;

FIG. 8 is a cross-sectional view of the front cap taken along line 8-8 in FIG. 7;

FIG. 9 is a front elevational view of the piston for use in the present invention;

FIG. 10 is a top view of the rubber plunger; and

FIG. 11 is a cross-sectional view of the plunger taken along line 11-11 of FIG. 10.

### DETAILED DESCRIPTION OF THE PREFERRED EMBODIMENTS

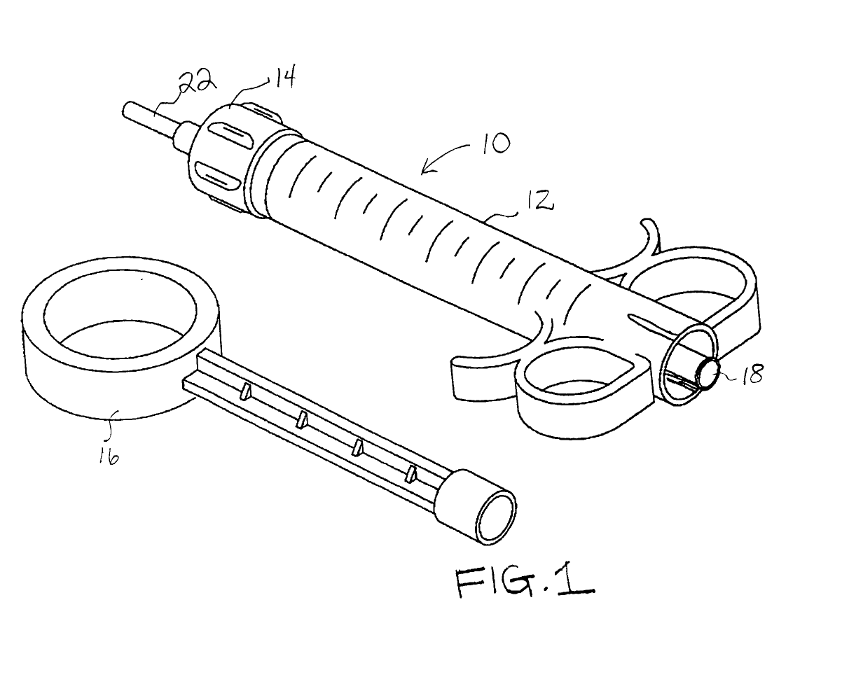

Referring to the drawings, FIG. 1 illustrates the syringe assembly of the present invention generally designated by the reference numeral 10. The main components of the syringe assembly include the syringe barrel 12, the front cap 14, the plunger handle 16 (shown disconnected from the syringe), the piston 18 connected to the rubber plunger 20 (shown in FIG. 10). The front cap 14 may have a cover 22, shown in FIG. 1, as may the piston 18 (see FIG. 2).

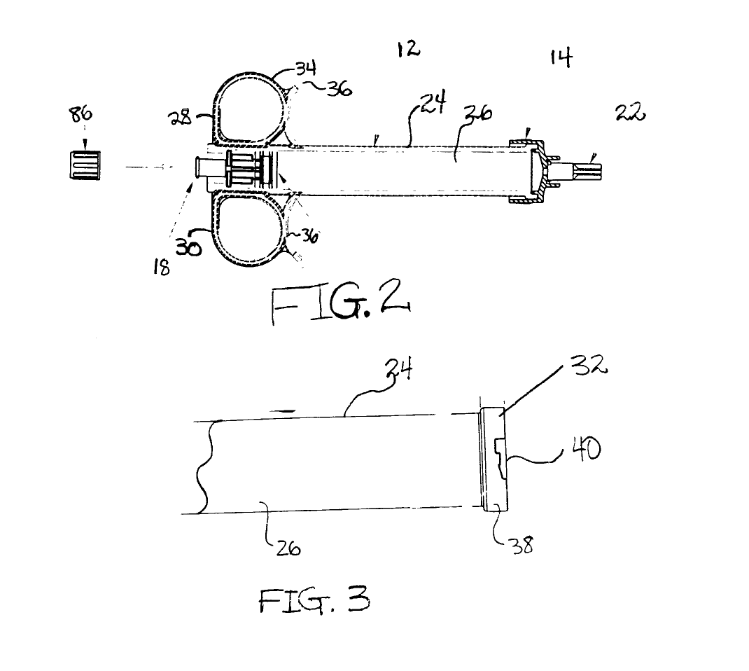

The construction of the barrel 12 can be understood with reference to FIGS. 1, 2, and 3. The barrel 12 includes a longitudinally extending portion 24, which defines an inner longitudinally extending cavity 26 for holding and dispensing a bone repair material (not shown). The barrel 12 preferably includes a pair of opposed finger grips 28,30 at one end and a bayonet mount 32 at the other end (See FIG. 3). Each figure grip 28,30 includes a D-shaped ring 34, and a curved finger rest 36 extending from the barrel 12 to allow the surgeon to grip the syringe assembly 10 while sliding the handle 16 and plunger 20 through the interior of the barrel 12. The bayonet mount 32 for the bayonet cap 14 on the front end of the barrel includes an outwardly extending curved lip 38 and a pair of L-shaped ribs 40 on its exterior portion on opposite sides of the barrel 12.

The front cap 14 shown in FIGS. 2, 7 and 8 includes an annular ring shaped bearing portion 42 which surrounds an opening 44 through which bone graft material may be expressed. The front cap 14 also includes a pair of channels 46,48 to accommodate the ribs 40 at the bottom of each channel 46,48 are slots 50, 52 to engage the ribs 40 surrounding the opening 44 is preferably a conical portion 54 (see FIG. 8) to help facilitate expression of bone implant material (not shown). The opening 44 leads into a conduit 56. Surrounding the conduit 56 is an annular wall 58 which extends from the syringe barrel 24 at a distance from the conduit 56. The annular wall 58 preferably includes threading (not shown) along its inside to engage the cover 22 (see FIG. 2). Additionally, the exterior of the annular wall 58 has a plurality of ridges 43 spaced about the periphery thereof to facilitate gripping of the front cap 14. Of course, other connection mechanisms can be used, including threaded, press fit, and luer lock.

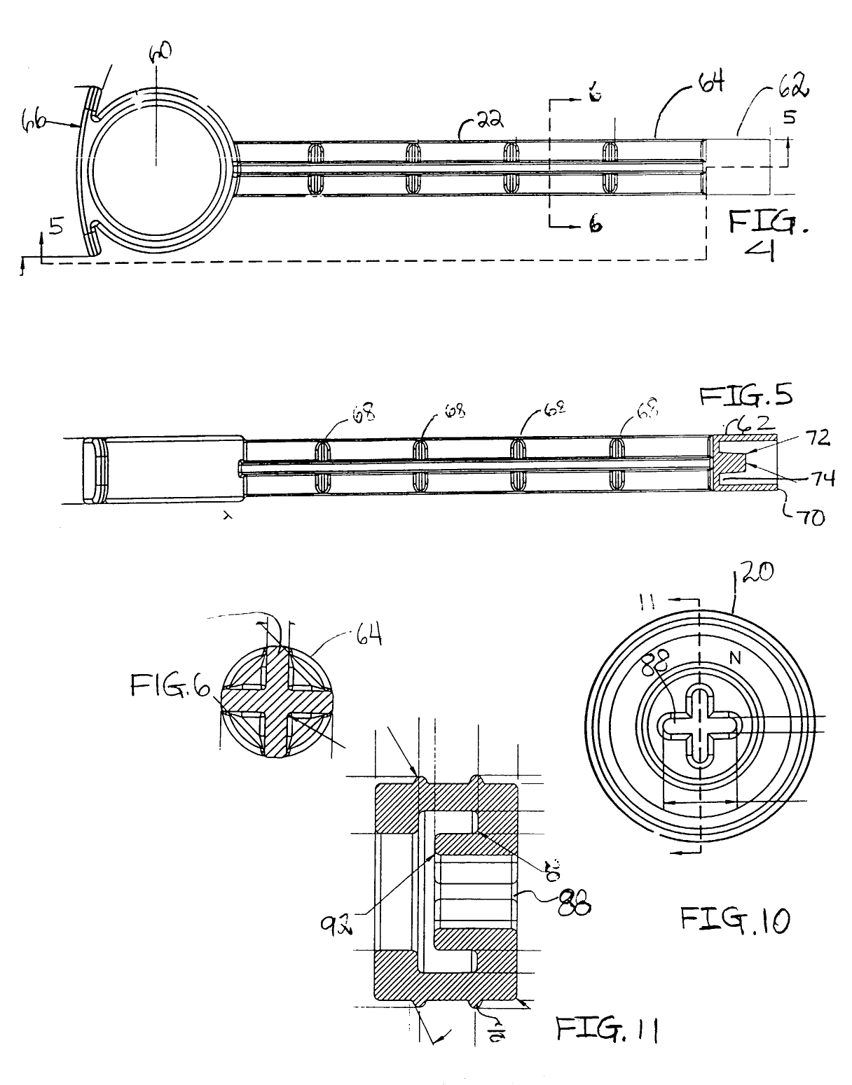

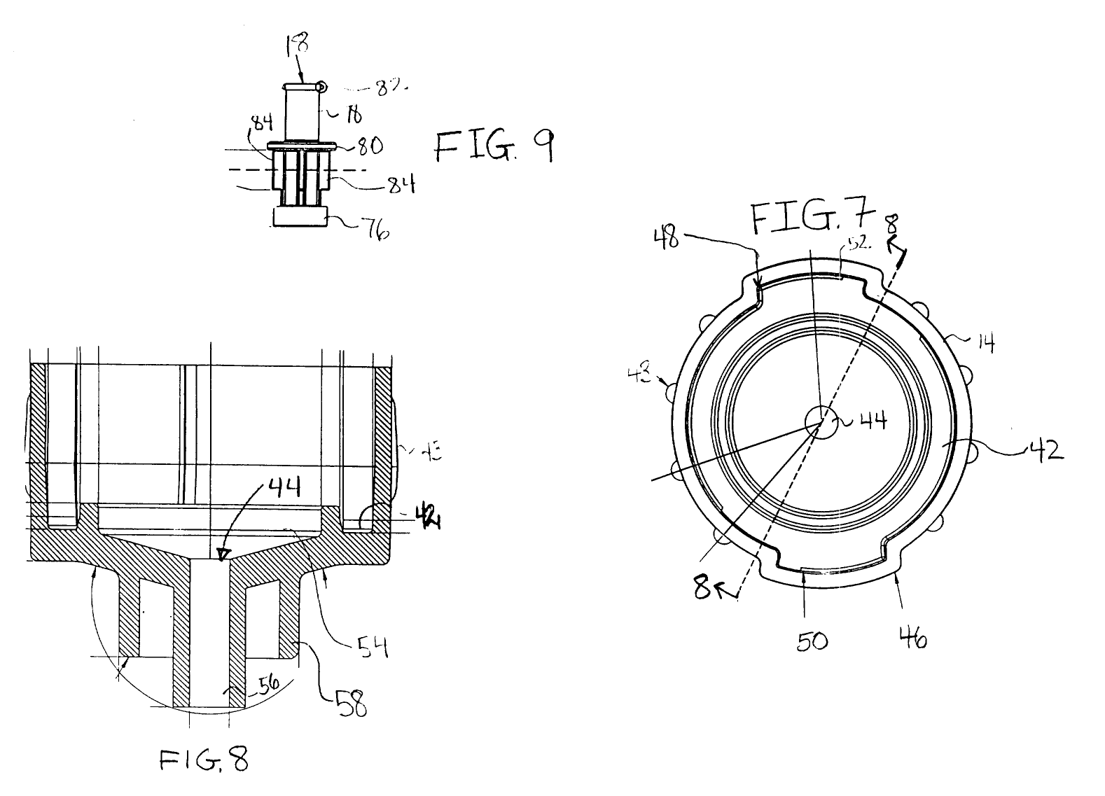

FIGS. 4, 5, and 6 illustrate the construction of the handle 22. The handle 22 includes a ring 60 at one end, a male, conical fitting 62 at the other end, and an intermediate shaft 64. The circular grip ring 60 includes a tangential bearing surface 66 which arcs to fit comfortably in the hand of the user and to enable even application of pressure to dispense the bone replacement or repair material contained in the barrel 12. The intermediate shaft 64 extends between the circular grip ring 60 and the male conical fitting 62. As shown in FIG. 6, it has a “plus shaped” cross-section and includes a plurality of reinforcements 68. The conical fitting 62 includes an annular wall 70 (see FIG. 5) and a truncated conical pin 72 centered therein. Annular wall 62 and the conical pin 72 define a bearing surface 74 therebetween.

The piston 18, shown in FIG. 9, includes an annular base 76 with a hollow shaft 78 extending upwardly therefrom. The hollow shaft 78 has a collar 80 positioned midway between the upper flange 82 and the annular base 76. A plurality of fins 84 extend outwardly from the hollow shaft 78 in a longitudinal direction around its periphery. A plastic protective cap 86 slides over the upper flange 82 to protect the opening to the hollow shaft 78 from dust and debris.

A rubber plunger 20 fits over the annular base 76 of the piston 18, as FIG. 11 illustrates. The plunger 20 includes an X-shaped opening 88 to allow air and excess fluid to escape from the barrel 12 or to permit fluid to be introduced into the inner cavity 26 defined by the barrel 12, and can include a permeable membrane to control permeation of various suspended particles while permitting passage of liquids.

The opening 88 connects to a passageway 90 through the body of the plunger 20. The interior of the plunger 20 has a C-shaped recess included therein to meet with the upper flange 82 of the piston 18. While the exterior has a plurality of spaced annular rings 90 to create a fluid tight, leak free seal with the wall of the barrel 12. With this construction, the syringe 10 allows liquids to pass in or out of the inner cavity 26 without losing any of the solids contained therein. Moreover, when the handle 16 connects to the piston 18, it seals the passageway 90 preventing leaks and preserving the sterile environment in the inner cavity 26. The handle 16 can attach to the piston 16 or even the plunger 14 through one of several conventional techniques including bayonet, threading, and press fit.

To assemble the components of the syringe assembly 10, the piston 18 is inserted into the opening 88 in the rubber plunger 20 until it seats in the C-shaped recess 92. The rubber plunger 20 with the piston 18 extending therefrom slides into the front opening of the syringe barrel 12. The front end of the syringe barrel 12 inserts into the front cap 14 with the ridges 43 aligned with the channels 46, 48, and twisted so that the prongs 40 engage the slots 50,52 to hold the cap 22 tightly in place. The cover 22 which protects the front opening 44 in the front cap 14 twist or screws into place. The handle 16 may be inserted into the syringe barrel 12 until it reaches the upper flange 82 of the piston 18, where it can be twisted gently to engage the piston 18. Alternatively, if the handle 16 is supplied in a kit, it can be disconnected from the piston 18, and a cap 86 can be press fit or twisted onto the upper flange 82 of the piston 18.

The syringe assembly of the present invention can be fabricated from a wide variety of materials, such as plastic, glass, or metal, or any combination thereof. The syringe barrel 12, front cap 14, and the piston 18 should preferably be made from clear, lipid resistant polycarbonate plastic, available as Bayer Part No. DP1-1805-1118. The plunger handle 16 can also be made from the foregoing lipid resistant polycarbonate material, but preferably should be colored white with Reed Omnicolor No. UN0005 in order to allow the position of the handle within the barrel to be readily detectable to the user. The rubber plunger 20 found on the tip of the piston 18 can preferably be made from 60 durometer dimethyl silicone (Precision Silicone PN PS2622), or other suitable natural or synthetic rubber material.

As mentioned, the syringe assembly 10 can be supplied as part of a kit for use in implanting solid, particulate bone graft material. Such solids can include, for example, autogenous morselized bone graft, autogenous bone graft strips, allograft chips, demineralized bone matrix in putty, gel, strip, or other forms, xenografts and fired bone. The solids can also be bone graft substitutes, such as hydroxyapatite, calcium carbonate, beta tricalcium phosphate, calcium sulfate or mineralized collagen. In addition, natural or synthetic polymers such as collagen particles, meshes, sponges, and gels, hyaluronic acid and derivatives thereof, liposomes or other natural biomaterials known as potential implants, or carriers of therapeutic agents, such as cytokines, growth factors, cells, antibiotics, analgesics, chemotherapeutic drugs, and the like.

As synthetic polymers, there can be alpha-hydroxy polyesters, including polylactic acid, polyglycolic acid and their copolymers, polydioxanone, as well as poly methyl methacrylate, separately, in mixture or in admixture with any of the foregoing therapeutic agents. Preferably, in kit form the syringe assembly is supplied with bone graft substitutes such as hydroxyapatite, calcium carbonate, or calcium sulfate.

The syringe also permits addition and mixing of a liquid, gel, or fluid substance to the inner chamber of the barrel either before, after (or instead of) addition of the solid component. Likewise, the syringe can be used to draw a gel or other fluid through a porous hydroxyapatite block, whether or not the syringe is used to help implant that block. The fluid substance may be drawn through the front opening in the syringe itself or the front cap 44 or injected through the opening in the plunger 88.

Among the fluids which can be added are sterile water, saline, blood, or blood components including plasma, platelet-rich plasma, buffy coat, autologous growth factors or other concentrated blood components, red blood cells, white blood cells or platelets in any combination, as well as cryoprecipitates. Other suitable and intended fluids include bone marrow, as well as growth factor solutions suspensions or gels, which include any of the well known growth factors such as Platelet-Derived Growth Factor (PDGF), Transforming Growth Factor Beta (TGF-β), Insulin-Like Growth Factor (IGF), Fibroblast Growth Factor (FGF), Epidermal Growth Factor (EGF), Vascular Endothelial Growth Factor (VEGF), Bone Morphogenetic Proteins (BMPs), and vectors for gene therapy. Further, cellular solutions, suspensions, and materials including osteoblasts, osteoprogenitor cells, chondroblasts, stem cells, or fibroblasts may also be used, as may solutions or suspensions containing other therapeutic agents such as antibiotics, analgesics, antithrombinolytics, or chemotherapeutic agents.

In use, the surgeon removes the syringe barrel 12 from the surrounding packaging (not shown) and inserts and twists the handle 22 into place so that it connects with the piston 18. The front cap 14 is removed to permit insertion of the desired solid phase material, after which the cap is replaced and locked into place (Although in some applications, the front cap need not be replaced). The desired liquid or fluid may be drawn through the front opening or inserted through the opening in the plunger (after removing the handle). The handle is reattached, and the plunger is moved toward the front opening in order to express excess fluid or air.

Various modifications will be apparent to those of ordinary skill in the art upon reading this disclosure. For example, the size and shape of the barrel and the front opening may be adjusted to accommodate different types of materials. Additional ports may be added to the sidewall to permit introduction of still other components to a solid liquid mixture or suspension. It is contemplated, however, that all such modifications, additions, and improvements be included within the scope and spirit of the invention and protected by the following claims.

What is claimed is:

1. A syringe assembly for dispensing a bone substitute material, comprising:
an elongated barrel having an outer wall, a front opening, and a rear opening, the outer wall defining a longitudinally extending cavity therein;
a cover for the front opening having a sealable opening therein to allow substances and fluids to enter and exit the longitudinally extending cavity;
a flexible, fluid tight plunger sized to slide in the longitudinally extending cavity to divide the longitudinally extending cavity into first and second portions, the plunger having a small opening for allowing passage of fluids from the longitudinally extending cavity without permitting solids to pass therethrough; and
a handle connected to the plunger for sliding the plunger toward or away from the front opening.

2. A syringe assembly in accordance with claim 1, wherein the detachable handle includes a gripping ring at one end.

3. A syringe assembly in accordance with claim 1, wherein the handle is detachable from the plunger and includes a connector for connecting the handle to the plunger.

4. A syringe assembly in accordance with claim 1, wherein the connector is a luer lock which connects the handle to the plunger.

5. A syringe assembly in accordance with claim 1, wherein the cover attaches to the elongated barrel with a bayonet lock.

6. A syringe assembly in accordance with claim 1, wherein the cover attaches to the elongated barrel with a luer lock.

7. A syringe assembly in accordance with claim 1, wherein the cover screws onto the elongated barrel.

8. A syringe assembly in accordance with claim 5, wherein the elongated barrel is cylindrical and has tabs on the exterior thereof to engage and hold the cover on the elongated barrel.

9. A syringe assembly in accordance with claim 1, wherein the elongated barrel is cylindrical and wherein the assembly additionally comprises a pair of gripping rings extending outward on opposite sides of the barrel adjacent the rear opening.

10. A syringe assembly in accordance with claim 9, wherein the longitudinally extending space contains particles of hydroxyapatite.

11. A syringe assembly in accordance with claim 1, wherein the plunger is artificial or synthetic rubber or silicone rubber.

12. A syringe assembly for dispensing a bone substitute material, comprising:
an elongated barrel having front and rear openings and an outer wall defining an inner cavity for holding the bone substitute material;
a cap on the front end of the barrel having a sealable opening therein for dispensing the bone substitute material;
a flexible, fluid tight plunger sized to slide longitudinally in the cavity to divide and seal first and second portions of the longitudinally extending interior space from one another, the plunger having a small opening therein for allowing passage of fluids into or from the longitudinally extending cavity without permitting solids to pass therethrough;
a piston connected to the plunger for connecting a handle to the plunger; and
a detachable handle connectable to the piston for sliding the plunger toward or away from the front opening.

13. A syringe assembly in accordance with claim 12 wherein the bone substitute material includes hydroxyapatite particles.

14. A kit for dispensing a bone substitute material comprising:
a syringe assembly in accordance with claim 13; and
a natural or synthetic bone substitute material.

15. A kit in accordance with claim 14 additionally comprising bone growth stimulating agent.

16. A kit in accordance with claim 15 wherein the bone growth stimulating agent is TGF-β or a bone morphogenetic protein.

17. A kit in accordance with claim 14 additionally comprising a bone growth stimulating agent and a blood component.

18. A method of dispensing a bone substitute material, comprising:
providing a syringe assembly in accordance with claim 1;
adding to the inner cavity an amount of a solid particulate bone substitute material;
adding into the inner cavity a fluid preparation including a bone growth or repair stimulating or enhancing agent such that the fluid mixes with the bone substitute material to form a fluid-bone substitute mixture; and
dispensing the fluid-bone substitute mixture by moving the plunger within the inner cavity toward the cover.

19. A method in accordance with claim 18 wherein the fluid is added to the inner cavity of through the opening in the plunger.
